(19) United States
(12) Patent Application Publication
(10) Pub. No.: US 2024/0261466 A1
(43) Pub. Date: Aug. 8, 2024

(54) Method for Producing a Biocompatible Implant, and Implant

(71) Applicant: Karl Leibinger Medizintechnik GmbH & Co. KG, Mühlheim/Donau (DE)

(72) Inventors: Adem Aksu; Frank Reinauer; Tobias Wolfram

(21) Appl. No.: 18/006,685

(22) PCT Filed: Aug. 6, 2021
(86) PCT No.: PCT/EP2021/071975

§ 371 Date: Apr. 23, 2024

(30) Foreign Application Priority Data
Aug. 7, 2020 (DE) 10 2020 210 038.1

(51) Int. Cl. A61L 27/18; A61L 27/54; A61L 27/56; B29B 9/04; B29B 9/12; B29C 43/00; B29C 43/52; B29K 71/00; B29K 105/00

(52) U.S. Cl. CPC A61L 27/18; A61L 27/54; A61L 27/56; B29B 9/04; B29B 9/12; B29C 43/003; B29C 43/006; B29C 43/52; A61L 2300/404; A61L 2430/02; B29K 2071/00; B29K 2105/251

(57) ABSTRACT

The present invention provides a method for producing a biocompatible implant and a corresponding biocompatible implant. The biocompatible implant (10) has an implant body (8) composed of a polyaryletherketone plastic, wherein the implant body (8) has at least one first porous section (12) and one second porous section (13), wherein the first porous section (12) and the second porous section differ with respect to their porosity.

The present invention relates to a method for producing a biocompatible implant. The invention further relates to a corresponding biocompatible implant.

Polyaryletherketones (PAEK) are high-performance plastics comprising a polymer chain of ether and ketone groups and phenylene units. They have good mechanical properties, even at relatively high temperatures and under changing loads, and with respect to their wear behavior. In addition, they exhibit very good compatibility as biocompatible implant material, and so they have already been used for a long time in the production of implants. DE 10 2016 110 501 B3 discloses an additive manufacturing process for implants, which are produced from a granular material by means of a laser sintering process.

The formation of new blood vessels is a crucial process for successful integration and fixation of an implant. However, in the case of the hitherto known plastics composed of polyaryletherketones, ingrowth of soft tissue and bone is not possible in a defined manner.

It is therefore an object of the present invention to specify a method for producing an implant and a corresponding implant that more easily allows ingrowth of soft tissue and bone and that offers increased strength.

This object is achieved by the method having the features of claim 1 and the implant as claimed in claim 11.

According to the invention, the method in question is provided in particular with the following steps:
S1) providing a plastics powder composed of a polyaryletherketone;
S2) heating and pressing the plastics powder to form at least one intermediate piece;
S3) mechanically comminuting the at least one intermediate piece to form a granular material; and
S4) thermally bonding the granular material in a mold to form an implant, wherein the granular material is exposed to a spatially inhomogeneous heat distribution during the thermal bonding, wherein the implant has an implant body having at least one first porous section and one second porous section, wherein the first porous section and the second porous section differ with respect to their porosity.

By means of the thermal bonding with exposure to an inhomogeneous heat distribution, it is thus possible to produce individual three-dimensional implants composed of a polyaryletherketone plastic with a particulate formulation of at least one granular material and having a plurality of porous sections differing with respect to their porosity. It is self-evident that it is also possible according to the invention to produce implants with multiple thermally bonded granular materials.

The porous and topographically individually defined implant as a product-by-process allows improved ingrowth of soft tissue and bone in an environment-specific manner. This extensive vascular ingrowth assists in transporting important cells deep into the implant, which cells fight infections and are thus capable of modifying inflammation. At the same time, the ingrowth of biological tissue increases the strength of the implant. The implant is primarily specifically fixed via the porous properties and via the macrostructural properties of the production process. The implant can therefore accordingly be regarded as both microstructured and macrostructured; it is not built up in layers, but manufactured as a one-piece component.

Advantageous embodiments and variants can be found in the dependent claims.

In an advantageous variant of the method according to the invention that allows differentiation of the product in a simple manner, the mold is heatable with a plurality of heating devices which are each activated separately during bonding of the granular material in order to produce the inhomogeneous heat distribution. The heating devices can, for example, be arranged in different spatial directions around the granular material to be thermally bonded. Furthermore, the inhomogeneity of the heat distribution can also be realized by time-dependent and output-related activation of the heating devices.

In another advantageous embodiment, the polyaryletherketone is a polyetheretherketone, in which two ether groups and one ketone group at a time follow one another, thereby making it possible to manufacture a particularly highly compatible implant with a balanced profile of properties.

In order to allow optimum heat distribution and input at the implant to be produced, the granular material comminuted from the intermediate piece can, in a convenient variant, be formed with particles having a round, rounded, partially angular or polygonal cross section. In this case, round is to be understood to mean not only a circular cross section, but also, for example, an oval or an elliptical cross section, or else an irregularly rounded cross section. In addition, the cross section can also be semicircular or polygonal in such a way that it appears approximately round. Conceivable polygonal cross sections with a small number of sides include, for example, quadrangular, pentagonal, hexagonal or octagonal cross sections. Furthermore, different mixtures of granular materials of differing cross section with regard to shape and size are for example also conceivable.

In a preferred development in which the porous sections of the implant have a certain degree of homogeneity, the at least one granular material comminuted from the intermediate piece can be comminuted with a uniformly round cross section. However, it is not necessary for the porous sections of the implant to be homogeneous.

Another preferred development can consist in subjecting the plastics powder to an antimicrobial treatment before the preparation of the granular material and/or admixing a bioactive component in the plastics powder before the preparation of the granular material, with the result that the granular material and later the implant can have an intrinsically antimicrobially active pore structure.

In order to be able to flexibly set the strength characteristics of the implant to be produced, the particle size of the granular material can be varied in an advantageous variant of the method, the granular material having a particle size of between 1 μm and 3000 μm.

The implant to be produced can, for example, be formed with a plurality of different layers, which can be formed either with a nonporous structure or with porous structures. To this end, in an advantageous variant of the method, the mold can be filled differentially, i.e., with a plurality of granular materials differing with respect to their particle size and/or particle shape, before the bonding of the at least one granular material. Here, the different granular materials can each form a layer having independent properties during the thermal bonding, though in turn two or more layers of the same original granular material are also conceivable in principle.

Owing to modifications of the particulate character of the forms of granular material and owing to admixtures of bioactive components and the differential filling of the forms of construction in the production process, the implant can also have a structured porosity in the sense of a vectorial pore distribution with differing bioactivity and the biological fixation of the implant can thus be achieved.

In another advantageous variant, for further variation of the properties of the implant, the mold can exert a specifiable pressure on the at least one granular material during the bonding of the at least one granular material, thereby making it possible for example to increase the shear strength of the implant.

In a preferred variant of the method, the mold can comprise a shaping means which forms a cavity within the structure of the individual three-dimensional implant during the thermal bonding of the granular material. The macroscopic structure of the implant body is thus designed in such a way that it can be used as a hollow chamber system for accommodating hydrogels or bioinks, for example. As a result, the implant body has a depot effect.

The shaping means can be a negative form of the cavity to be formed, for example a bulge formed on the mold, from which the implant must subsequently be demolded; also conceivable is a balloon-like structure which has been filled with a fluid, for instance, and is removed from the subsequently resulting cavity after the bonding of the granular material. Lastly, also conceivable is a shaping means in the form of a rotary mold.

During its production, the implant according to the invention is three-dimensionally adapted to an accommodating structure in the body of a human body, the implant body of the adapted implant being provided with a first porous section and with a second porous section, the two porous sections differing with respect to their porosity. Because of the porous sections within its structure, an at least semiporous implant with optimized osteointegration and possible bioactivity is established. The relevant sections of the implant are preferably provided with an interconnecting pore structure, and the open, uninterrupted cavities formed as a result mean that said sections are configured to allow ingrowth of supply structures for new bone structure to be formed. Porosity can also be introduced only partially in the implant.

Owing to the balanced properties thereof, an advantageous development of the implant is formed from polyetheretherketone (PEEK).

In an advantageous embodiment of the implant, which embodiment allows variation of the properties of the implant, the implant can have a layered structure having a plurality of layers, at least one of which comprises the porous sections. For example, the implant for instance can be provided with a solid (nonporous) layer and a (possibly partially) porous layer comprising the differently porous sections. It is also possible that the porous sections themselves each form such a layer.

Furthermore, the implant can be provided with, for example, an outer layer which at least partially delimits said implant, the structure of which outer layer differs from one or more inner layers, it being possible for the outer layer to be especially solid. Here, the implant can furthermore comprise at least one inner layer adjoining the outer layer, which inner layer can be provided with sections of differing partial porosity.

In an advantageous embodiment, the implant body can have a total porosity with value ranges between 10% and 80%, in particular sections having a porosity of between 10-20%, 20-40%, 40-60% and/or 60-80%, thereby making it possible to achieve flexible strength characteristics on the implant.

The porous sections can conveniently have different pore sizes, it being possible for the pore sizes to vary in a range from 1 μm to 3500 μm. A porosity gradient can be formed using different pore sizes, thereby making it possible to improve cell migration properties at the implant and the diffusion of nutrients and waste products at the implant. Said gradient can be configured to extend in a layer-faithful manner or to extend over layers, and can be a curve with or without discontinuities in a plot.

In an advantageous embodiment which, for example, can better assist healing processes, the implant can have within its structure at least one cavity which is configured to accommodate a bioactive substance.

Said substance can be housed in the cavity either before or after the introduction of the implant into the accommodating structure of the human body. What can thus be formed is a porous implant having a macroscopic placeholder structure for accommodating, for instance, inorganic calcium phosphate-based materials, which can be introduced as an injectable substance and improve tissue regeneration.

In an advantageous development, the porous, patient-specific implant, for example composed of PEEK, can be provided with placeholder structures in the size range from 500 μm to 2 mm for accommodating bioactive hydrogel and/or bioink formulations for optimizing cell therapy applications. Furthermore, the porous implant can utilize the properties of the cavity to serve as a “biocage” for cell culture systems. However, other sizes of the cavity are also conceivable.

In another development of the implant according to the invention, the cavity can be provided with an interface section which connects the cavity to the exterior of the implant. Before or after the implant is inserted into the accommodating structure, at least one substance is injectable into the cavity via the interface section.

In yet another development, the interface section can conveniently be provided with a guide means for application, accommodation or alignment of an injection means. For the purpose of guidance, the interface section can for example be cylindrical, and its edge can be provided with a step in the manner of a flange for application and alignment of the injection means. However, other configurations are also conceivable.

In preferred developments, the substances to be stored in the cavity, assigned to the implant, can be provided as at least one hydrogel, at least one bioink, at least one inorganic calcium phosphate-based material or as a cell culture.

Improved take of bone tissue can be achieved by means of an advantageous embodiment of the implant which has at least one roughened section on its surface. Such an active implant surface due to the significant enlargement thereof can, for example, be achieved by sandblasting or acid etching, since the microroughness produced promotes incorporation of the implant.

A hydrophilic surface section has similar advantages in the take of tissues. On the other hand, a hydrophobic surface section may possibly influence the adherence properties and thus the accumulation of bacteria. In a convenient development, the implant can therefore, for example, be at least regionally hydrophobic on a first side and at least regionally hydrophilic on a second side.

In one development, in order to be able to individually adapt the implant to the accommodating structure, the implant can be flexibly shapable, in particular flexibly shapable by heating in a saline solution.

The above configurations and developments can be combined with one another as desired where appropriate. Further possible configurations, developments and implementations of the invention also encompass combinations of features of the invention that have not been explicitly mentioned, which features have been described above or will be described below with regard to the exemplary embodiments. In particular, a person skilled in the art will also add individual aspects as improvements or additions to the basic forms of the present invention.

The invention will be more particularly elucidated below on the basis of exemplary embodiments in the figures of the drawing.

In the figures:

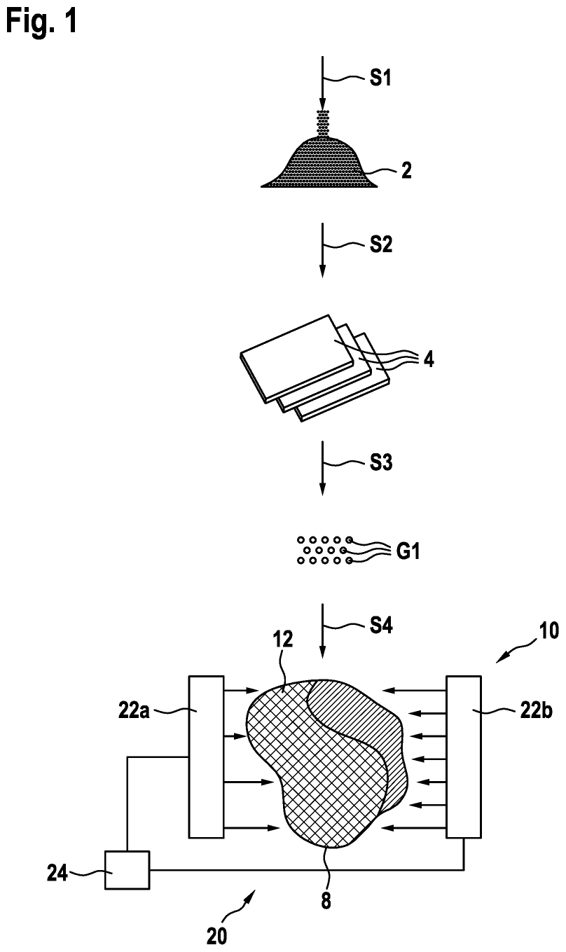

FIG. 1 shows a schematic flow chart of one embodiment of the method according to the invention for producing the implant comprising the method steps S1 to S4;

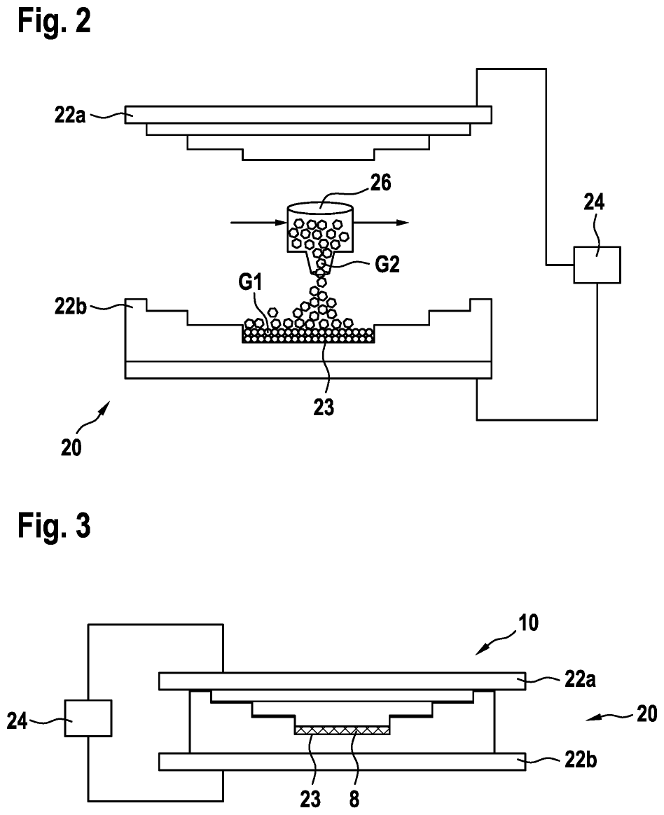

FIG. 2 shows a cross-sectional view of an open mold that is being filled with granular material;

FIG. 3 shows a side view of the mold according to FIG. 2 in the closed state after the production of the implant body;

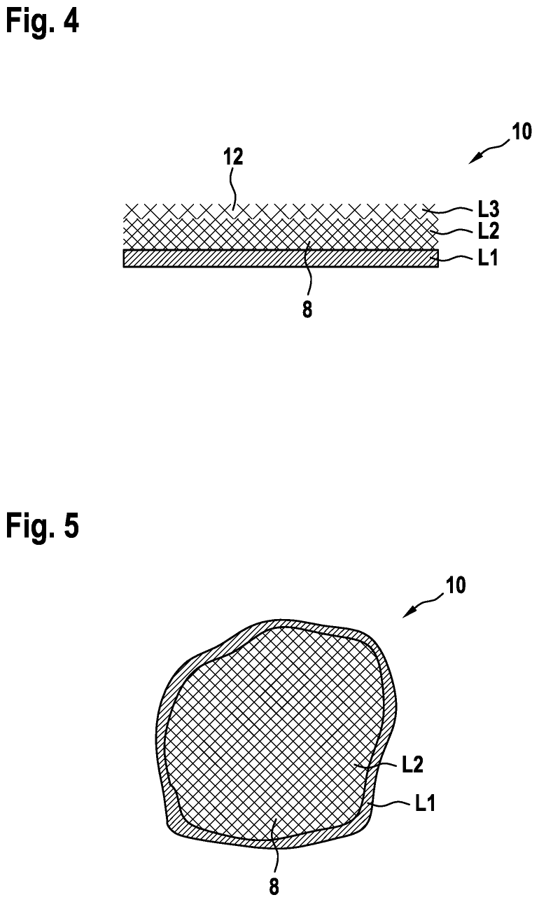

FIG. 4 shows a sectional side view of a first embodiment of an implant body having a plurality of layers and differently porous sections;

FIG. 5 shows a sectional side view of a further embodiment of an implant body having a solid outer layer;

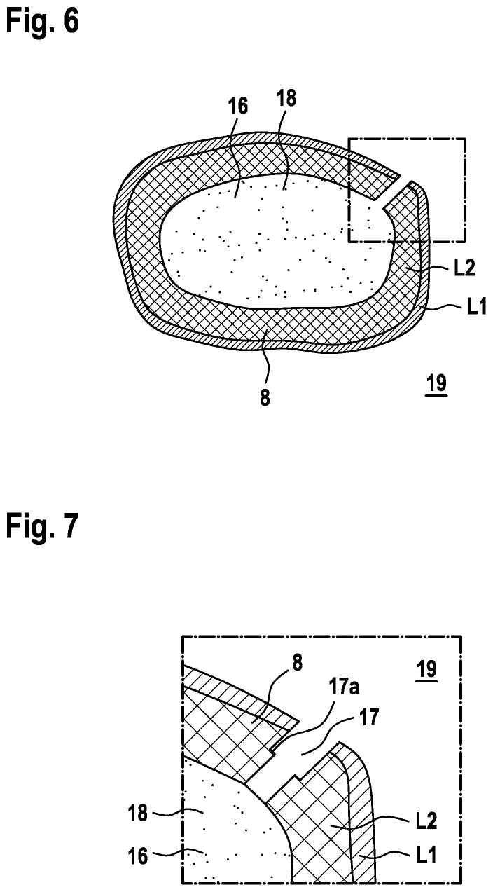

FIG. 6 shows a sectional side view of a further embodiment of an implant body in the structure of which a cavity is formed;

FIG. 7 shows a sectional side view of a partial detail of the embodiment according to FIG. 6;

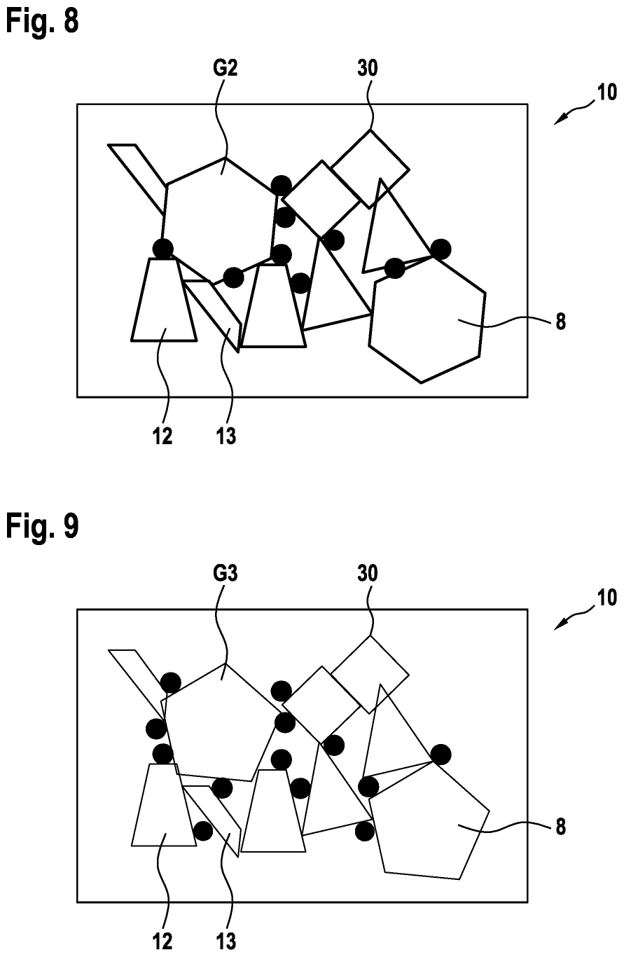

FIG. 8 shows a sectional side view of one embodiment of the implant body of the implant having a porous surface and porous sections composed of a hexagonal granular material with admixtures; and

FIG. 9 shows a sectional side view of one embodiment of the implant body of the implant having a porous surface and porous sections composed of a pentagonal granular material with admixtures.

In all the figures, identical or functionally identical elements and devices have been provided with the same reference signs, unless otherwise stated.

FIG. 1 shows a schematic flow chart of one embodiment of the method according to the invention for producing the implant comprising the method steps SI to S4.

According to FIG. 1, there is first provided a plastics powder 2 in the form of a PEEK powder 2 (step S1). The free-flowing plastics powder 2 is then pressed by means of a sintering-like process, as identified by step S2. This forms one or more one-piece/coherent intermediate pieces 4. In particular, the intermediate pieces 4 are achieved by pressing and simultaneously heating the plastics powder 2, the intermediate pieces 4 ultimately forming rectangular plates. The temperature of the intermediate pieces 4 during this sintering/primary shaping of the intermediate pieces 3 is always below the decomposition temperature of the plastics powder 2 used (or, in the case of multiple plastics materials, below the decomposition temperature of the material component of the plastics powder 2 used that has the lowest melting point). Preferably available for producing an intermediate piece 4 is a negative form, into which the plastics powder 2 is first filled and then heated up and also compressed with exposure to a pressing force, with the result that an intrinsically solid structure is formed in the form of the intermediate pieces 4.

Following the production of the intermediate pieces 4, each intermediate piece 4 is comminuted in a defined manner according to step S3. The intermediate pieces 3 are comminuted into particles to form a granular material G1. The particles of the granular material Gl have a substantially uniform shape, which is realized by the specific implementation of the mechanical comminution. In this exemplary embodiment, round particles, in the form of spherical particles or particles with an oval cross section, are produced.

According to step S4, what then follows is thermal bonding of the granular material 4 of set shape to form the individual, one-piece three-dimensional implant body 8. In this exemplary embodiment, the exposure to a spatially inhomogeneous heat distribution from a mold 20 serves for thermal bonding, with the result that two porous sections 12, 13 of differing porosity are formed on the implant body 8 (using a mold not depicted here).

In this method, after step S4 has been carried out, the implant body 8 has substantially the finished shape of the implant 10 to be produced. Here, the implant 10 is typically in the form of an implant 1 for osteosynthesis or fracture treatment. The thermal bonding is carried out in such a way that the implant 10/implant body 8 has a porous structure, preferably an open-pore structure. Alternatively, closed-pore structures are also realizable.

Furthermore, it can be seen in FIG. 1 that two heating devices 22a, 22b, which are operated by a controller 24, are arranged on the mold 20. The inhomogeneity of the heat distribution to which the granular material G1 is exposed is achieved by the two separately activated heating devices 22a, 22b, the heating device 22a emitting heat of a temperature T1 and the heating device 22b emitting heat of a temperature T2, with the result that sections 12, 13 having differing porosity are formed on the implant body 8.

FIG. 2 shows a cross-sectional view of an open mold that is being filled with granular material.

According to FIG. 2, the mold 20 is filled with two granular materials G1, G2 by a filler 26 in order to carry out the method according to the invention. The first granular material Gl having a round cross section is already arranged in the mold bed 23 arranged on the lower heating device 22b, and the filler 26 is now differentially filling the mold bed 23 with another granular material G2, the granular shape of which has a different cross section, namely a hexagonal cross section.

FIG. 3 shows a side view of the mold according to FIG. 2 in the closed state after the production of the implant body.

In FIG. 3, it can be seen that the mold 20 is closed and the implant body 8 of the implant 10 has been formed as a result of exposure to the inhomogeneous temperature distribution from the heating devices 22a, 22b, controlled by the controller 24, which implant body 8 can be demolded from the mold 20 after the latter has been opened.

FIG. 4 shows a sectional side view of a first embodiment of an implant body having a plurality of layers and differently porous sections.

In FIG. 4 is an implant body 8 having a plurality of layers L1, L2, L3, of which layer LI, which is lowermost for the observer, is solid and thus nonporous. In the present case, said layer LI forms an outer layer that at least partially delimits the implant body 8 and differs structurally from further, inner layers L2, L3. In the upward direction for the observer, there follow said further layers L2, L3 of differing porosity, layer L2 having a porosity in the range of 10-20% and layer L3 having a porosity in the range of 20-40%. As a result, layers L2 and L3 form porous sections 12, 13 of the implant body 8 of differing porosity and thus form a porosity gradient.

FIG. 5 shows a sectional side view of a further embodiment of an implant body having a solid outer layer.

The implant body 8 has a porous outer region as first section 13, which is formed with a lower porosity than that of a porous inner region as second section 12.

FIG. 6 shows a sectional side view of a further embodiment of an implant body in the structure of which a cavity is formed.

What can be seen in FIG. 6 is a cross section of an implant body 8, which again has a porous outer region as a section 13, which encloses another section 12 as an inner region of higher porosity. Within its structure, the implant body 8 is provided with a cavity 16 having an oval cross section. Accommodated in the cavity is a bioactive substance 18 in the form of a hydrogel which promotes tissue regeneration. The cavity 16 has a transverse extent of a few millimeters.

FIG. 7 shows a sectional side view of a partial detail of the embodiment according to FIG. 6.

The detail in FIG. 7 shows that the cavity 16 of the implant body 8 is provided with an interface section 17 which connects the cavity 16 to the exterior 19 of the implant 10. What is created as a result is access to the cavity 16, via which the bioactive substance 18 can be deposited in the cavity at a desired moment. Likewise shown is the arrangement on the interface section 17 of a guide means 17a which, as a flange-like cross-sectional constriction of the interface section, allows the application, accommodation or alignment of an injection means, such as a cannula or the like, that is not shown.

FIG. 8 shows a sectional side view of one embodiment of the implant body of the implant having a porous surface and porous sections composed of a hexagonal granular material with admixtures, and FIG. 9 shows a sectional side view of one embodiment of the implant body of the implant having a porous surface and porous sections composed of a pentagonal granular material with admixtures.

According to FIG. 8, a hexagonal polyetheretherketone (PEEK) granular material G2 was used, and according to FIG. 9, a pentagonal PEEK granular material G3 was used.

In addition, the granular materials have each been admixed with different additives before the thermal bonding, a ceramic component in the case of the implant body 8 in FIG. 8 and silver and copper as metals in the case of the implant body 8 in FIG. 9.

If the powders for producing the granular materials have each undergone a corresponding pretreatment, the resultant pore structures are each intrinsically bioactive, in the present case antimicrobially active. Both implant bodies 8 have a porous surface 30, i.e., they are, as already mentioned, open-pore structures. Their porous sections 12, 13 form interconnecting pore structures with pores in the order of magnitude of a few 100 μm, which support the ingrowth of biological tissue and increase the strength of the implant 10. The implant 10 is primarily specifically fixed via its porous properties and via the macrostructural properties of its production process.

Although the present invention has been described above on the basis of preferred exemplary embodiments, it is not restricted thereto, but is modifiable in a variety of ways. In particular, the invention can be altered or modified in many ways without departing from the essence of the invention.

1. A method for producing a biocompatible implant, comprising the following steps:
providing a plastics powder composed of a polyaryletherketone;
heating and pressing the plastics powder to form at least one intermediate piece;
mechanically comminuting the at least one intermediate piece to form a granular material; and
thermally bonding the granular material in a mold to form an implant, wherein the at least one granular material is exposed to a spatially inhomogeneous heat distribution during the thermal bonding, wherein the implant has an implant body having at least one first porous section and one second porous section, wherein the first porous section and the second porous section differ with respect to their porosity.

2. The method as claimed in claim 1, wherein the mold is heated with a plurality of heating devices which are each activated separately during the thermal bonding of the granular material in order to produce the inhomogeneous heat distribution.

3. The method as claimed in claim 1, wherein the polyaryletherketone is polyetheretherketone.

4. The method as claimed in claim 1, wherein the granular material comprises particles having a round, rounded, partially angular or polygonal cross section.

5. The method as claimed in claim 1, wherein the granular material comprises particles having a round cross section.

6. The method as claimed in claim 1, wherein, before the preparation of the granular material, the plastics powder is subjected to an antimicrobial treatment and/or a bioactive component is admixed in the plastics powder.

7. The method as claimed in claim 1, wherein the granular material has a particle size of between 1 μm and 3000 μm.

8. The method as claimed in claim 1, wherein the mold is filled with a plurality of granular materials differing with respect to their particle size and/or particle shape before the thermal bonding of the granular material.

9. The method as claimed in claim 1, wherein the mold exerts a specifiable pressure on the at least one granular material during the thermal bonding of the granular material.

10. The method as claimed in claim 1, wherein the mold comprises a shaping means which forms a cavity within the implant body during the thermal bonding of the granular material.

11-24. (canceled)
